(19) United States
(12) Patent Application Publication
(10) Pub. No.: US 2010/0204254 A1
(43) Pub. Date: Aug. 12, 2010

(54) Use of statins in the prevention and treatment of radiation injury and other disorders associated with reduced endothelial thrombomodulin

(75) Inventors: Martin K. Hauer-Jensen, Little Rock, AR (US); Louis M. Fink, Little Rock, AR (US); Jawahar Lal Mehta, Little Rock, AR (US); Junru Wang, Little Rock, AR (US); Jacob Joseph, Little Rock, AR (US)

Correspondence Address: Benjamin Aaron Adler, Ph.D., J.D., 8011 Candle Ln, Houston, TX, 77071, US

(73) Assignee: The University of Arkansas for Medical Sciences, Little Rock, AR (US)

(21) Appl. No.: 12/657,915

(22) Filed: Jan. 29, 2010

Related U.S. Application Data
(63) Continuation of application No. 10/658,045, filed on Sep. 9, 2003, now abandoned.
(60) Provisional application No. 60/459,511, filed on Mar. 31, 2003.
(60) Provisional application No. 60/409,787, filed on Sep. 11, 2002.

(51) Int. Cl. A61K 31/505; A61K 31/225; A61K 31/366; A61K 31/40; A61K 31/404

(52) U.S. Cl. 514/275; 514/548; 514/460; 514/423; 514/419; 435/375

(57) ABSTRACT

The present invention discloses statins (3-hydroxy-3-methylglutaryl coenzyme A reductase inhibitors) consistently and significantly increased endothelial cell thrombomodulin protein and functional activity. Statins also abrogated the downregulation of thrombomodulin that occurs in response to radiation injury. These results indicate that preserving or restoring endothelial thrombomodulin expression and function by statins may be useful in a variety of disorders associated with widespread endothelial dysfunction such as sepsis, adult respiratory distress syndrome, and normal tissue radiation injury.

### CROSS-REFERENCE TO RELATED APPLICATIONS

This continuing application claims benefit of priority under 35 U.S.C. §120 of pending non-provisional U.S. Ser. No. 10/658,045, filed September 2003, which claims benefit of priority under 35 U.S.C. §119(e) of provisional application U.S. Ser. No. 60/459,511, filed Mar. 31, 2003, now abandoned, and provisional application U.S. Ser. No. 60/409,787, filed Sep. 11, 2002, now abandoned, the entirety of all of which are hereby incorporated by reference.

### FEDERAL FUNDING LEGEND

This invention was produced in part using funds obtained through grant R01CA83719 from the National Institutes of Health. Consequently, the federal government has certain rights in this invention.

### BACKGROUND OF THE INVENTION

1. Field of the Invention

The present invention relates generally to the pharmacology and medical therapeutics of 3-hydroxy-3-methylglutaryl coenzyme A reductase inhibitors (HMC-CoA reductase inhibitors, statins). More specifically, the present invention relates to the uses of statins in disorders associated with reduced endothelial thrombomodulin.

2. Description of the Related Art

The transmembrane glycoprotein thrombomodulin is located on the luminal surface of endothelial cells and plays a pivotal role in coagulation-anticoagulation homeostasis by forming a complex with thrombin. In normal situations, the thrombin-thrombomodulin complex inhibits thrombin-induced conversion of fibrinogen to fibrin and activates protein C. Activated protein C acts as a potent anticoagulant by combining with protein S to inactivate Factors Va and VIIIa of the blood coagulation pathway and by binding thrombin. In situations accompanied by endothelial cell loss or dysfunction, a lack of thrombomodulin causes thrombin to activate the coagulation cascade and generate fibrin clots, thus resulting in a strongly prothrombotic environment.

Human thrombomodulin contains 559 amino acid residues and has similarities to the LDL receptor. The molecule contains both O-glycosylation and N-glycosylation sites as well as having 1-2 molecules of chondroitin sulfate bound to it. There are six repeated epidermal growth factor homologous domains and the amino terminal domain has homology to lectin-like proteins. DNA sequences for human, bovine, rat and mouse thrombomodulin have been cloned and there is extensive interspecies homology. Thrombomodulin expression is suppressed by inflammatory products such as interleukin 1, tumor necrosis factor and endotoxin, whereas interleukin 4, retinoic acid and agents which increase cAMP such as forskolin have been shown to up-regulate thrombomodulin activity in endothelial cells in culture.

In addition to the lining cells of arteries, veins, capillaries and lymphatics, thrombomodulin has been found in several other types of cells. Thrombomodulin has been found in mesothelial cells, meningeal lining cells, synovial cells, syncytiotrophoblasts, megakaryocytes, platelets and squamous cell carcinoma cells. Thrombomodulin has been used to immunochemically stain a variety of vascular tumors and choriocarconomas (Fink et al., 1993).

The importance of thrombomodulin deficiency is well documented in a variety of disorders associated with widespread endothelial dysfunction such as sepsis, adult respiratory distress syndrome, and normal tissue radiation injury. Currently, replacement therapy with recombinant thrombomodulin or recombinant activated protein C are the only methods by which the specific thrombomodulin functional defect can be influenced. However, administration of recombinant proteins is costly and associated with significant logistical and pharmacological problems. The present invention provides an alternative approach of increasing thrombomodulin expression and function by using a pharmacologically safe and effective agent statins (3-hydroxy-3-methylglutaryl coenzyme A reductase inhibitors).

The prior art is deficient in effective treatment of disorders associated with reduced endothelial thrombomodulin. The present invention fulfills this longstanding need and desire in the art.

### SUMMARY OF THE INVENTION

The present invention discloses statins (3-hydroxy-3-methylglutaryl coenzyme A reductase inhibitors) consistently and significantly increased endothelial cell thrombomodulin protein and functional activity. Statins also abrogated the downregulation of thrombomodulin that occurs in response to radiation injury and in response to the inflammatory cytokine TNF-alpha. In view of the limited treatment options for disorders associated with thrombomodulin deficiency, the low cost of statins and their very favorable side effect profile make statins very appealing as new therapeutic agents for disorders associated with thrombomodulin deficiency.

The present invention provides a method of increasing cell surface thrombomodulin expression and function through the use of compounds widely referred to as “statins”.

In another embodiment of the present invention, there is provided a method of using statins to prevent or treat a disorder associated with endothelial dysfunction and thrombomodulin deficiency.

In another embodiment of the present invention, there is provided a method to prevent or treat injury of normal tissues that occurs during or after radiation therapy of cancer.

In yet another embodiment of the present invention, there is provided a method of preventing or treating a radiation-exposed individual, comprising the step of administering to a subject an effective amount of 3-hydroxy-3-methylglutaryl coenzyme A reductase inhibitor.

In yet another embodiment of the present invention, there is provided a method of treating an individual having a neoplastic disease, comprising the steps of: administering to said individual an effective amount of 3-hydroxy-3-methylglutaryl coenzyme A reductase inhibitor; and treating said individual with radiation therapy.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1F show that statins increase thrombomodulin in three endothelial cell lines. Incubation for 24 hours with atorvastatin (10 mM) increases cell surface thrombomodulin antigen (FIGS. 1A-1C) and thrombomodulin activity (FIGS. 1D-1F) in human coronary endothelial cells (HCAEC), human umbilical vein endothelial cells (HUVEC) and EA.hy926 endothelial cells.

FIGS. 2A-2D show the time-dependent effect of atorvastatin on endothelial cell thrombomodulin. EA.hy926 endothelial cells were incubated for various times with 10 mM atorvastatin. FIG. 2A: steady-state thrombomodulin mRNA; FIG. 2B: thrombomodulin protein; FIG. 2C: cell surface thrombomodulin; FIG. 2D: thrombomodulin activity.

FIGS. 3A-3D show that treatment of EA.hy926 cells with atorvastatin different concentrations of atorvastatin for 24 hrs increases endothelial cell thrombomodulin activity in a dose-dependent manner. The dose-dependence is highly statistically significant (p<0.0001). FIG. 3A: steady-state thrombomodulin mRNA; FIG. 3B: thrombomodulin protein; FIG. 3C: cell surface thrombomodulin; FIG. 3D: thrombomodulin activity.

FIG. 4 shows that treatment of EA.hy926 cells with different concentrations of simvastatin for 24 hrs increases endothelial cell thrombomodulin activity in a dose-dependent manner. The dose-dependence is highly statistically significant (p<0.0001).

FIGS. 5A-5B show the effects of statin on normal and irradiated endothelial cells. FIG. 5A: thrombomodulin antibody binding sites (determined by flow cytometry). FIG. 5B: thrombomodulin activity (protein C activation assay). The graphs show that statin applied 1 hour before radiation greatly increases endothelial cell surface thrombomodulin protein and thrombomodulin activity, and that statin more or less reverses the effect of irradiation on thrombomodulin activity. All measurements are performed 24 hours after irradiation. All differences between control cells and statin-treated cells are significant (p<0.0001)

FIG. 6 shows a Western blot of EA.hy926 cell lysates. Atorvastatin-treated cells show a prominent increase in thrombomodulin protein, both in irradiated and non-irradiated cells.

FIG. 7 shows a simplified diagram of the mevalonate pathway, showing the various substrates and enzyme inhibitors used. HMG-CoA=3-hydroxy 3-methylgiutaryl coenzyme A; FPP=farnesyl pyrophosphate; GGPP=geranylgeranyl pyrophosphate; FTI=farnesyl transferase inhibitor; GGTI=geranylgeranyl transferase inhibitor; ZGA=zaragozic acid.

FIGS. 8A-8D show the inhibition of atorvastatin's effect on endothelial cell thrombomodulin by mevalonic acid. EA.hy926 endothelial cells were incubated with 10 mM atorvastatin, 500 mM mevalonate, or both. FIG. 8A: steady-state thrombomodulin mRNA; FIG. 8B: thrombomodulin protein; FIG. 8C: cell surface thrombomodulin; FIG. 8D: thrombomodulin activity.

FIGS. 9A-9B show an increase in endothelial cell thrombomodulin in response to nitric oxide donors and inhibition of atorvastatin's effect on endothelial cell thrombomodulin by a nitric oxide scavenger. FIG. 9A: effect of incubation of EA.hy926 endothelial cells with a rapid nitric oxide donor (SIN-1) and a slow nitric oxide donor (PAPA-NONOate) on thrombomodulin activity. FIG. 9B: effect of nitric oxide scavenging on the atorvastatin-induced increase in thrombomodulin activity in EA.hy926 cells.

FIG. 10 shows the effect of ATORVASTATIN™ and SIMVASTATIN™ on endothelial thrombomodulin activity (protein C activation assay) in human intestinal microvascular cells.

### DETAILED DESCRIPTION OF THE INVENTION

3-hydroxy-3-methylglutaryl coenzyme A reductase inhibitors (statins) potently inhibit cholesterol biosynthesis and reduce total cholesterol, LDL cholesterol, triglycerides, and apo B. In addition to prominent lipid-lowering effects, statins are also known to have a number of other effects. These non-lipid-lowering functions of statins include anti-inflammatory effects, antiproliferative effects, effects on the actin cytoskeleton, and anticoagulant and fibrinolytic effects that have been attributed primarily to an increase in plasminogen activator and a decrease in tissue factor, plasminogen activator inhibitor-1, and endothelin-1.

The toxicity profile of statins is very benign and the side effects are usually mild and reversible. Statins are commonly used in patients with hyperlipidemia, but potential uses of these drugs in other disorders are largely unexplored. While statins are known to have anti-inflammatory effects and modest anticoagulant properties, the effects of statins on thrombomodulin are not known.

The present invention shows that statins consistently and very significantly increase endothelial cell thrombomodulin protein and functional activity, and that statins abrogate the downregulation of thrombomodulin that occurs in response to endothelial cell injury. Results disclosed herein indicate that statins can be used as new therapeutic agents to increase thrombomodulin expression in disorders associated with thrombomodulin deficiency.

The importance of the thrombomodulin deficiency is well documented in a variety of disorders associated with widespread endothelial dysfunction such as sepsis, adult respiratory distress syndrome, and normal tissue radiation injury.

Recent clinical trials and other reports of the treatment of patients with severe sepsis have focused on the coagulation system, since activation of the coagulation system and depletion of endogenous anticoagulants are frequently noted in patients with severe sepsis and septic shock (Bernard et al., 2001; Faust et al., 2001; Warren et al., 2001). Organ dysfunction is often associated with diffuse microthrombus formation in these patients. However, high-dose antithrombin III therapy had no effect on 28-day all-cause mortality in adult patients with severe sepsis and septic shock when administered within 6 hours after the onset (Warren et al., 2001). A study of the expression of thrombomodulin and the endothelial protein C receptor in the dermal microvasculature of children with severe meningococcemia and purpuric or petechial lesions demonstrated that protein C activation is impaired. There was a marked reduction in the expression of thrombomodulin and endothelial protein C receptor on the endothelium of both thrombosed and nonthrombosed dermal vessels in children with early meningococcal disease.

The finding that plasma levels of activated protein C were low or undetectable in children with meningococcal sepsis, as well as the failure of activated protein C levels to rise after administration of unactivated protein C concentrate suggests that the reduction in endothelial expression of thrombomodulin and endothelial protein C receptor results in the impairment of protein C activation. A phase III clinical trial for treatment of sepsis with recombinant human activated protein C resulted in a statistically significant increase in survival (Bernard et al., 2001). These findings are consistent with down-regulation of the endothelial thrombomodulin-endothelial protein C receptor pathway, and demonstrate the potential importance of the thrombomodulin-activated protein C pathway in sepsis and organ dysfunction.

Acute lung injury leading to the acute respiratory distress syndrome (ARDS) is a serious complication of both trauma and sepsis. The mortality for acute respiratory distress syndrome in these conditions often exceeds 50%. The coagulation system and the immune system combine to produce lung injury characterized by edema, hemorrhage, microvascular thrombosis and neutrophil infiltration. Studies in an animal model demonstrated that systemic inflammatory state produced by intraperitoneal zymosan (an animal model of adult respiratory distress syndrome such as occurs in individuals with severe sepsis) produced a decrement in lung tissue thrombomodulin. Not only did this reduction occur in the organ suffering the most severe injury but it was also detected in the specific areas of the lung that showed evidence of injury with edema and inflammation. This finding is consistent with previous findings in patients dying of pneumonia (Albertson et al., 2001). In both circumstances, the lung injury is heterogeneous. In areas where normal lung architecture is preserved, lung thrombomodulin is densely distributed throughout the alveolar capillaries. In regions of lung damage, the thrombomodulin is markedly diminished. These findings indicate that downregulation of thrombomodulin may lead to a hypercoagulable endothelium, increased microvascular thrombosis and subsequent lung injury, and enhancement of thrombomodulin expression may have pathophysiological significance in human acute respiratory distress syndrome.

There is ample evidence to suggest that radiation causes a state of local hypercoagulability. Hence, radiation induces a plethora of microvascular alterations, including endothelial cell swelling, increased permeability, interstitial fibrin deposition, and development of platelet-fibrin thrombi. At the cellular level, radiation causes increased apoptosis, increased permeability, inflammatory cell adhesion and emigration, decreased fibrinolysis, and increased prothrombotic properties by increased expression of tissue factor and von Willebrand factor (vWF), and decreased expression of prostacyclin and thrombomodulin. These observations are consistent with the notion that radiation increases the prothrombotic properties of endothelial cells and that endothelial dysfunction may mediate, contribute to, or sustain some aspects of normal tissue radiation toxicity.

Intestinal toxicity (radiation enteropathy) is a major dose-limiting factor in radiation therapy of abdominal and pelvic tumors. Depending on the time of presentation relative to radiation therapy, radiation enteropathy is classified as acute or delayed. Acute radiation enteropathy is a result of epithelial barrier breakdown and mucosal inflammation. In contrast, delayed radiation enteropathy, which may present clinically many years after radiation therapy, is characterized by vascular sclerosis and progressive intestinal wall fibrosis. Microvascular injury is believed to be a key factor in the pathogenesis of radiation fibrosis in many organs, including intestine, and likely responsible for the chronic and progressive nature of delayed radiation injury.

Correlative evidences from clinical studies strongly suggest a role for thrombomodulin in the pathogenesis of radiation fibrosis. First, in small bowel-resection specimens from patients undergoing operations for radiation enteropathy, there was a sixfold reduction in the number of thrombomodulin-positive submucosal vessels compared to normal intestine (Richter et al., 1997). Second, analysis of specimens from patients who had received adjuvant radiation therapy before undergoing resection of rectal cancer revealed that the radiation-induced deficiency of microvascular thrombomodulin was a premorbid phenomenon, i.e., thrombomodulin was deficient before the development of discernible evidence of radiation enteropathy (Richter et al., 1998). Moreover, results from an animal study demonstrated that localized fractionated irradiation of the intestine caused a consistent, time-dependent, dose-dependent decrease in thrombomodulin on microvascular endothelium, and that the severity of thrombomodulin deficiency correlated with structural, cellular, and molecular aspects of early and delayed radiation toxicity (Wang et al., 2002). Together these findings raise the clinically important possibilities that preserving or restoring endothelial thrombomodulin may protect against normal tissue toxicity in patients undergoing radiation therapy of cancer.

In addition to the above described disorders, other situations and disorders where there is evidence for a role of thrombomodulin include aortocoronary and peripheral vascular bypass procedures, various autoimmune diseases, inflammatory bowel disease, and various conditions associated with adverse tissue remodeling. Statins could potentially be of clinical benefit in many or all of these conditions. Furthermore, thrombomodulin is essential for normal embryonic development, and its absence causes embryonic lethality in mice before development of a functional cardiovascular system and expression of the thrombin gene (Healy et al., 1995).

The present invention is directed towards a method of increasing cell surface thrombomodulin expression and function, comprising the step of administering a 3-hydroxy-3-methylglutaryl coenzyme A reductase inhibitor to a cell such as endothelial cell.

The present invention also provides a method of using 3-hydroxy-3-methylglutaryl coenzyme A reductase inhibitors to treat a disorder associated with endothelial dysfunction and thrombomodulin deficiency. Representative disorders associated with endothelial dysfunction and thrombomodulin deficiency include sepsis, adult respiratory distress syndrome, and tissue radiation injury. In general, the inhibitor is administered orally or intravenously or parenterally. Representative 3-hydroxy-3-methylglutaryl coenzyme A reductase inhibitors include pravastatin and its sodium salt, simvastatin, lovastatin, rosuvastatin, atorvastatin and fluvastatin.

The present invention also provides a method of treating a radiation-exposed individual, comprising the step of administering to a subject an effective amount of 3-hydroxy-3-methylglutaryl coenzyme A reductase inhibitor. A person having ordinary skill in this art would readily recognize the optimal doses and routes of administration for this method of the present invention. Preferably, inhibitor is administered orally or intravenously or parenterally. 3-hydroxy-3-methylglutaryl coenzyme A reductase inhibitors are well known in the art. Representative 3-hydroxy-3-methylglutaryl coenzyme A reductase inhibitors include, but are not limited to, pravastatin and its sodium salt, simvastatin, lovastatin, atorvastatin, rosuvastatin and fluvastatin. This method of the present invention would be useful in treating an individual exposed to a therapeutic amount of radiation. For example, 3-hydroxy-3-methylglutaryl coenzyme A reductase inhibitors could be beneficially used to treat an individual that received a therapeutic radiation treatment for a cancerous or pre-cancerous condition. In addition, this method of the present invention would be useful in treating an individual exposed to non-therapeutic ionizing of radiation. For example, 3-hydroxy-3-methylglutaryl coenzyme A reductase inhibitors could be beneficially used to treat an individual exposed to radiation in a radiation accident, in nuclear warfare, in an event of radiation terrorism (a “dirty bomb” or other explosive device) or in a space flight. The method of the present invention would also be useful if applied prophylactically, i.e., before radiation exposure occurs. Situations where prophylactic use would be applicable include individuals scheduled to receive radiation therapy of cancer, individuals who will be exposed to ionizing radiation, e.g., astronauts; and individuals who are at increased risk of being exposed to ionizing radiation by accidents, acts of terrorism or acts of war, e.g., radiation workers, workers at nuclear power plants, individuals in areas of heightened terrorism as well as civilians and military personnel in areas of conflict.

The present invention is also directed to a method of treating an individual having a neoplastic disease, comprising the steps of: administering to said individual an effective amount of 3-hydroxy-3-methylglutaryl coenzyme A reductase inhibitor; and treating said individual with radiation therapy. Typically such an inhibitor is administered orally or parenterally. Representative inhibitors include pravastatin and its sodium salt, simvastatin, lovastatin, atorvastatin, rosuvastatin and fluvastatin.

When used in vivo for therapy, a drug/compound useful in the methods of the present invention is administered to the patient or an animal in therapeutically effective amounts, i.e., amounts that preserve or restore endothelial thrombomodulin expression and function, or amounts that eliminate or reduce the toxicity during and following radiation therapy. It may be administered in a solid or liquid form. For example, it may be administered orally, preferably in an enterosoluble preparation, or rectally in a suppository or in an enema. The dose and dosage regimen of statins will depend upon the radiation dose(s) being administered to the patient e.g., the therapeutic index, the patient, the patient's history and other factors. A single dose of statin administered will typically be in the range of about 0.05 to about 5 mg/kg of patient weight, whereas the typical single dose for small animals such as dog and cat will be somewhat higher, i.e., in the range of about 1 to about 50 mg/kg of body weight. The dose and dosing schedule can be optimized for effectiveness while balanced against negative effects of treatment. See Remington's Pharmaceutical Science, 17th Ed. (1990) Mark Publishing Co., Easton, Pa. and later editions; and Goodman and Gilman's: The Pharmacological Basis of Therapeutics, 8th Ed (1990) Pergamon Press and later editions.

Examples of pharmaceutically acceptable carriers are water, saline, Ringer's solution, dextrose solution, and 5% human serum albumin. Nonaqueous vehicles such as fixed oils and ethyl oleate may also be used. Liposomes may be used as carriers. The pharmaceutical composition may contain minor amounts of additives such as substances that enhance isotonicity and chemical stability, e.g., buffers and preservatives. The drug of the present invention will typically be formulated in such vehicles at concentrations of about 0.001 mg/ml to 100 mg/ml so that the final dose is about 0.05 to 5 mg/kg of patient body weight or about 1 to 50 mg/kg of animal (i.e. dog, cat, etc.) body weight.

The following examples are given for the purpose of illustrating various embodiments of the invention and are not meant to limit the present invention in any fashion:

### Example 1

### Reagents

Atorvastatin and simvastatin were from Pfizer (New York, N.Y.) and Merck Laboratories (Whitehouse Station, N.J.). Mevalonate, farnesyl-pyrophosphate (FPP), geranylgeranyl-pyrophosphate (GGPP), squalene, zaragozic acid A (ZGA), 3-(2-Hydroxy-2-nitroso-1-propylhydrazino)-1-propanamine (PAPA-NONOate), 3-Morpholinosydnonimine hydrochloride (SIN-1), and 2-Phenyl-4,4,5,5-tetramethylimidazoline-1-oxyl 3-oxide (PTIO) were from Sigma Chemical Co. (St. Louis, Mo.); geranylgeranyl transferase I inhibitor (GGTI-298) and farnesyl protein transferase inhibitors I and II (FPTI-I and FPTI-II) were from Calbiochem (San Diego, Calif.). Recombinant hirudin and human protein C were from American Diagnostica (Greenwich, Conn.); chromogenic substrate for activated protein C was from Chromogenix (Milano, Italy); and human recombinant tumor necrosis factor-E\ (TNF-£\) from R&D Systems (Minneapolis, Minn.).

### Example 2

### Endothelial Cells

In vitro studies were performed using human endothelial cell lines. Human umbilical vein endothelial cells (HUVECs) and human coronary artery endothelial cells (HCAECs) were from Clonetics (San Diego, Calif.). EA.hy926 endothelial cells are an immortalized cell line that is a hybrid between human umbilical vein endothelial cells (HUVECs) and a lung (type II pneumocyte) adenocarcinoma cell line. This cell line, originally obtained from Dr. Cora-Jean S. Edgell in the Department of Pathology at the University of North Carolina, is widely used for endothelial cell studies.

Human umbilical vein endothelial cells were cultured in EGM-2 Bulletkit medium containing endothelial cell basal medium-2 (EBM-2) and EGM-2 SingleQuots (hEGF, hydrocortisone, hFGF-B, VEGF, R³-IGF-1, ascorbic acid, Gentamicin, Amphotericin-B, fetal bovine serum, and heparin). Human coronary artery endothelial cells were cultured in EGM-2 MV BulletKit medium containing EBM-2 with hEGF, hydrocortisone, hFGF-B, VEGF, R³-IGF-1, ascorbic acid, Gentamicin, Amphotericin-B, and fetal bovine serum. EA.hy926 cells were cultured in Dulbecco's modified Eagle medium containing 4.5 g/L glucose, 10% fetal calf serum, penicillin (50 U/ml) and streptomycin (50 mg/ml), L-glutamine, and hypoxanthine-aminopterin-thymidine supplement (100 μmol/L hypoxanthine, 0.4 μmol/L aminopterin, and 16 μmol/L thymidine). Cultures were maintained at 37° C. in a humidified atmosphere with 5% CO₂. Experiments were performed with cells in early confluence.

### Example 3

### Fluorogenic Probe RT-PCR

Steady-state TM mRNA levels were measured using quantitative real-time RT-PCR (Dr. G. Shipley, Quantitative Genomics Core Laboratory, University of Texas Health Science Center, Houston, Tex.). Human thrombomodulin and b-actin fluorogenic oligonucleotide probes and primers were designed from Genbank sequences, using Primer Express software (Applied Biosystems, Foster City, Calif.) and synthesized by Biosource International (Camarillo, Calif.). Total RNA was extracted using TRIZOL Reagent (Invitrogen, Carlsbad, Calif.) and the RNA samples were treated with RNAse-free DNAse I (Promega, Madison, Wis.). cDNA was synthesized from total RNA in 10 μl total volume, consisting of 6 μl RT master mix (Invitrogen, Carlsbad, Calif.) and a 4-μl RNA sample (30 ng/μl), using a thermocycler (MJR, Waltham, Mass.) for 30 minutes at 50° C. followed by 72° C. for 10 min. Samples were measured in triplicate. An assay-specific sDNA standard spanning a 5-log range and appropriate controls were included. Forty ml of a PCR master mix was pipetted directly onto each well of the cDNA plate utilizing a Biomek 2000 robotic workstation (Beckman, Fullerton, Calif.), and amplified (95° C. for 1 min; 40 cycles of 95° C. for 12 sec, and 60° C. for 1 min) using the 7700 Sequence Detector (Applied Biosystems). The data were normalized to b-actin and presented as molecules of TM transcript/molecules of b-actin transcript×100 (% b-actin).

### Example 4

### Western Blotting

Cells were harvested with trypsin/EDTA and lysed in lysis buffer (10 mM Tris, pH7.4, 150 mM NaCl, 1 mM EDTA, IGPAL CA630, 1 mM PMSF). Protein concentration was measured with the BCA assay (Pierce, Rockford, Ill.). Proteins (20 μg/per lane) were resolved by NuPAGE 4-10% Bis-tris gel electrophoresis (Invitrogen, Carlsbad, Calif.) and electrotransferred onto nitrocellulose membranes. Membranes were probed with primary mouse anti-human thrombomodulin antibody (American Diagnostica), followed by application of a secondary horseradish peroxidase-conjugated (HRP) anti-mouse IgG antibody (American Diagnostica). A mouse anti-human b-actin antibody (either from Sigma or Santa Cruz Biotechnology) was used for protein loading control. HRP signals were detected with an enhanced chemiluminescence (ECL) detection system (Amersham Pharmacia Biotech). Band densities were determined from X-Ray film using SigmaGel software (Jandel Scientific, San Rafael, Calif.).

### Example 5

### Flow Cytometry

Cells were examined for surface antigen expression by flow cytometry, using specific phycoerythrin-conjugated mouse antibodies against human TM (CD-141), a general endothelial cell marker (PECAM [CD-31]), and markers of stimulated endothelial cells (tissue factor [CD-142] and P-selectin [CD-62]) (BD Biosciences, San Diego, Calif.). Cell viability was assessed using 7-amino-actinomycin D (7-AAD). Phycoerythrin-conjugated mouse IgG was used as negative control. A total of 1×10⁵ cells were used for each analysis. Cells were harvested, stained with appropriate phycoerythin-conjugated antibodies, fixed in 1% paraformaldehyde, and analysis was performed using a FACSCalibur Flow Cytometer (BD Immunocytometry System, San Jose, Calif.), QuantiBRITE-PE beads (BD Biosciences, San Diego, Calif.), and CELLQuest software. Antigen levels were expressed as antibody binding sites (ABS).

### Example 6

### Protein C Activation Assay

TM activity was assessed by measuring protein C activation in early confluent cells grown on 96-well plates. Cells were washed twice with PBS and incubated with 0.5 mM protein C and 1 nM thrombin (60 min at 37° C., 60 ml total volume) to generate activated protein C. Excess thrombin was quenched with hirudin (20 ml, 0.2 ATu/ml). The amount of activated protein C generated was measured by monitoring hydrolysis of chromogenic substrate S-2366 at 5-min intervals at 405 nm in a microplate reader (Bio-TEK Instruments, Winooski, Vt.). The results were expressed as mean OD slope values (DOD/Dt).

### Example 7

### Statistical Analysis

Statistical analyses were performed with NCSS 2002 (NCSS, Kaysville, Utah). Differences among treatment groups were assessed with analysis of variance using Duncan's or Dunnett's post-hoc multiple range tests as appropriate. Two-sided statistical tests were used throughout.

### Example 8

### Atorvastatin and Simvastatin Increase the Expression and Activity of TM in Three Human Endothelial Cell Types

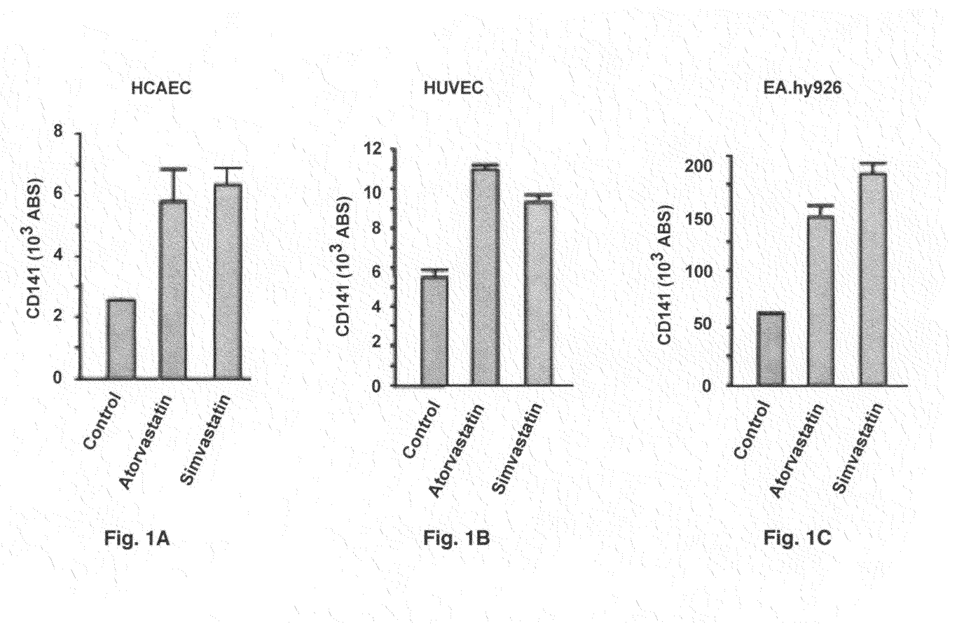

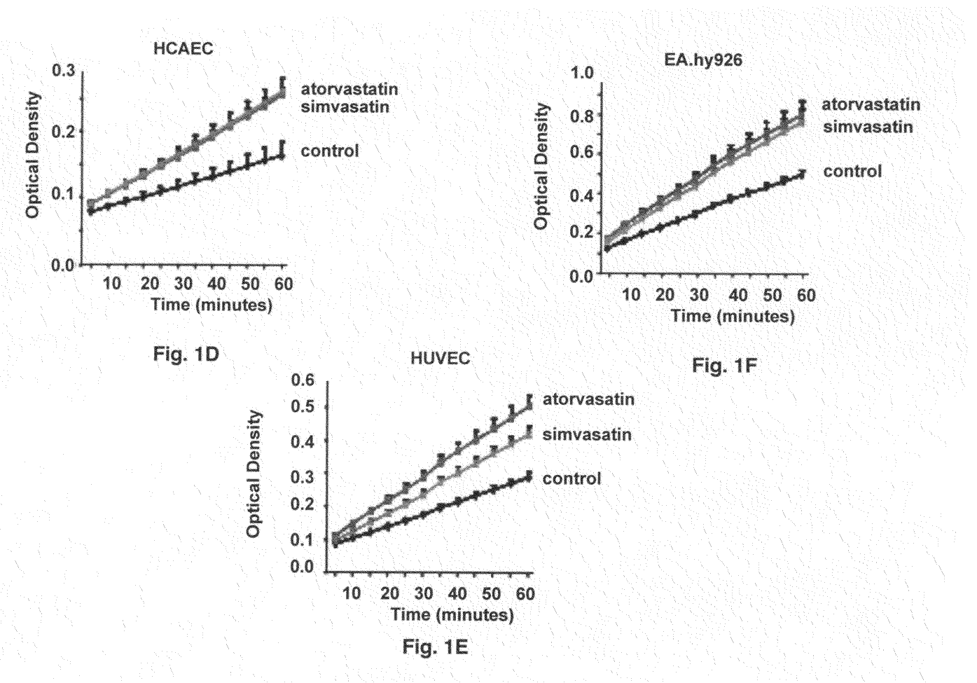

The effects of two different statins (atorvastatin and simvastatin) on three different endothelial cell types (HUVEC, HCAEC, and EA.hy926) were examined by flow cytometric analysis and protein C activation assay. Exposure of endothelial cells to 10 mM atorvastatin or simvastatin for 24 hours increased cell surface TM antigen and TM activity 2- to 3-fold in all three endothelial cell types (FIGS. 1A-1F). The two statins upregulated thrombomodulin to a similar extent. EA.hy926 cells (1C, 1F) expressed more constitutive thrombomodulin than HUVECs (FIG. 1B, 1E) and HCAECs (1A-1D) and exhibited the main features of normal, unstimulated endothelium. EA.hy926 cells were therefore used for the subsequent experiments.

Flow cytometric characterization of EA.hy926 cells revealed that the cell line express high levels of endothelial cell antigens CD-31 (PECAM) and CD-141 (thrombomodulin), but low levels of antigens expressed by stimulated endothelial cells, CD-62 (P-selectin) and CD-142 (tissue factor) (Table 1). As expected, control fibroblasts were low in CD-31, CD-62, and CD-141, but expressed high levels of CD-142. Comparison of the immortalized EA.hy926 cell line with standard HUVECs revealed that the 2 cell lines were similar in antigen expression, but that EA.hy926 expressed higher levels of thrombomodulin than HUVECs (Table 2). These data show that EA.hy926 cells have the characteristics of normal, quiescent (non-stimulated) endothelial cells.

The functional activity of thrombomodulin was assessed by measuring the ability of the endothelial cells to activate protein C. All samples and reagents were diluted in APC assay diluent (20 mM Tri-HCl, pH7.4, 100 mM NaCl, 2.5 mM CaCl₂, 0.5% BSA). Exponentially growing EA.hy 926 endothelial cells were seeded into 96 well plates in triplicate (2×10⁴ per well). Cells were allowed to attach and grow overnight, and then washed twice with PBS and incubated for 60 min in 60 μl total volume at 37° C. with 0.5 μM protein C and 1 nM thrombin to generate activated protein C. Excess thrombin was blocked with a superstoichiometric amount of hirudin (20 μl, 0.16 U/μl, 570 nM). Generation of active protein C was determined by using a chromogenic substrate S-2366 and absorbance at 405 nm was measured by using a Vmax kinetic microplate reader.

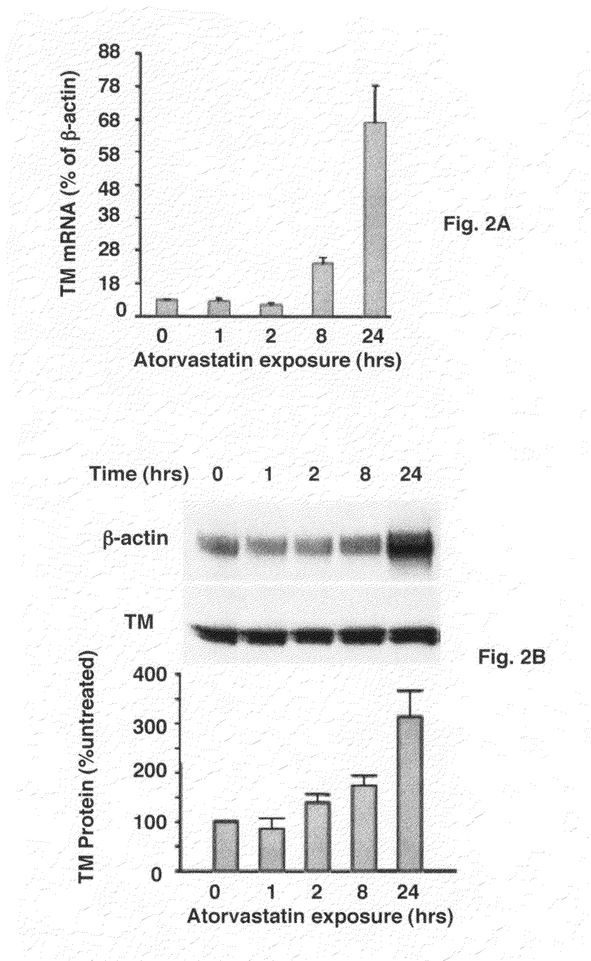

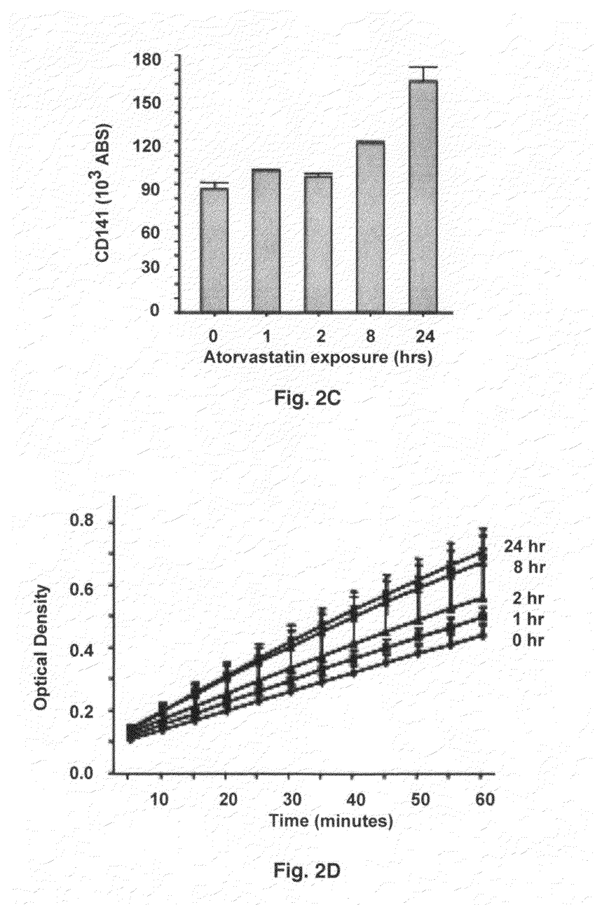

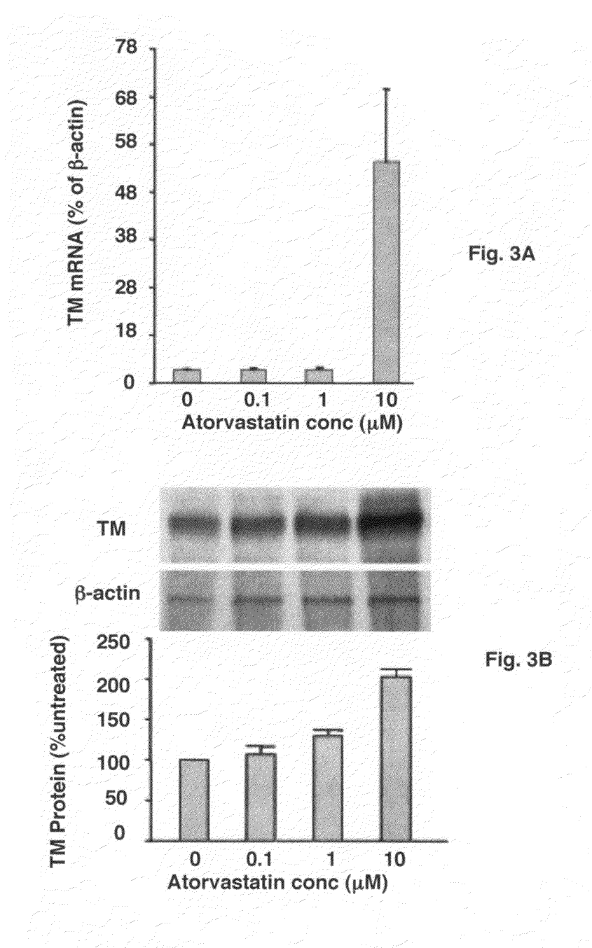

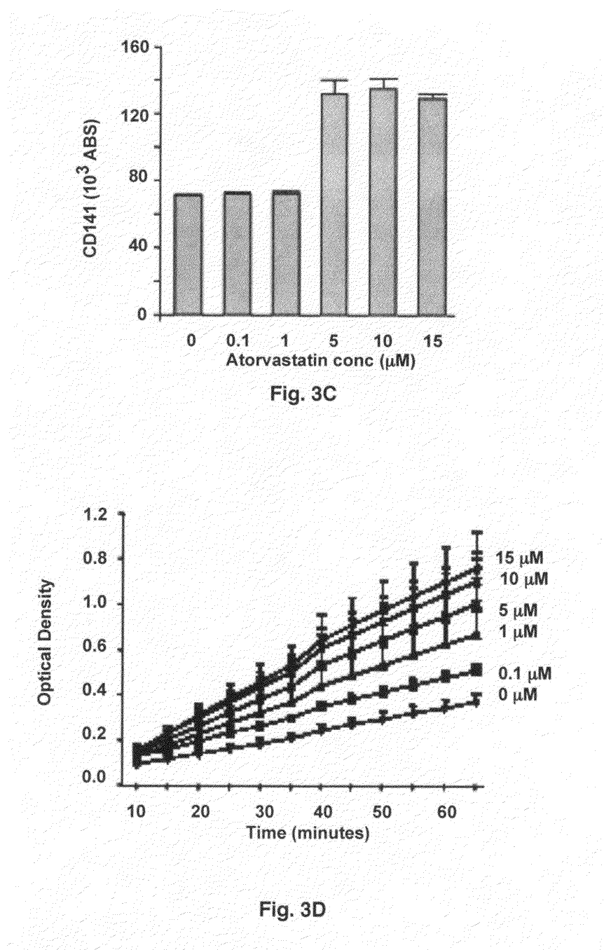

FIGS. 1A-1F show that 3 different statins enhanced in vitro thrombomodulin activity of endothelial cells, as determined by the protein C activation assay. Treatment with atorvastatin or simvastatin for 24 hours increased endothelial cell thrombomodulin activity in a dose-dependent manner (FIGS. 2A-2D, 3A-3D). The dose-dependence is highly statistically significant (p<0.0001). Treatment with atorvastatin for 24 hrs also increased endothelial cell surface thrombomodulin expression in a dose-dependent manner (FIGS. 3A-3D).

TABLE 1
Flow Cytometric Analysis of EA.hy926 Cells

| | EA.hy926 | Fibroblast |
|---|---|---|
| | (% positive) | (% positive) |
| Background (IgG control) | 1.2 ± 0.1 | 1.02 ± 0.02 |
| CD-31 (PECAM) | 87.8 ± 2.3 | 1.00 ± 0.08 |
| CD-62P (P-Selectin) | 1.6 ± 0.1 | 1.04 ± 0.08 |
| CD-141 (thrombomodulin) | 99.9 ± 0.06 | 1.05 ± 0.04 |
| CD-142 (tissue factor) | 1.4 ± 0.07 | 99.67 ± 0.06 |

TABLE 2
Comparison of EA.hy926 and HUVEC Surface Antigen Expression

| | EA.hy926 | HUVEC |
|---|---|---|
| | (% positive) | (% positive) |
| CD-62P (P-Selectin) | 1.6 ± 0.1 | 1.9 ± 0.1 |
| CD-141 (thrombomodulin) | 99.9 ± 0.06 | 85.6 ± 0.8 |
| CD-142 (tissue factor) | 1.4 ± 0.07 | 1.6 ± 0.7 |
| | EA.hy926 | HUVEC |
| | (10³ binding sites) | (10³ binding sites) |
| CD-141 (thrombomodulin) | 101.0 ± 2.3 | 11.1 ± 0.5 |
| CD-142 (tissue factor) | 1.8 ± 0.2 | 1.4 ± 0.1 |

### Example 9

### Atorvastatin-Induced Upregulation of TM is Time- and Concentration-Dependent

EA.hy926 cells were incubated with 10 mM atorvastatin for 0-24 hr and thrombomodulin mRNA levels (FIG. 2A), protein (FIG. 2B), surface antigen (FIG. 2C), and activity (FIG. 2D) were examined. Exposures to atorvastatin for 8- or 24 hours caused a 3-fold and >10-fold increase in thrombomodulin transcript, respectively. This was accompanied by a 2- to 3-fold increase in thrombomodulin protein, cell surface thrombomodulin antigen, and protein C activation at 24 hours. Incubation beyond 24 hours did not further increase thrombomodulin (data not shown). Incubation of EA.hy926 cells for 24 hours with atorvastatin concentrations of 0.1-15 mM revealed a prominent dose-dependent increase in mRNA (FIG. 3A) and cellular thrombomodulin protein (FIG. 3B), cell surface thrombomodulin antigen (FIG. 3C), and cell surface thrombomodulin activity (FIG. 3D). The concentration dependence was particularly evident for the functional assay.

### Example 10

### Simvastatin-Induced Upregulation of TM is Time- and Concentration-Dependent

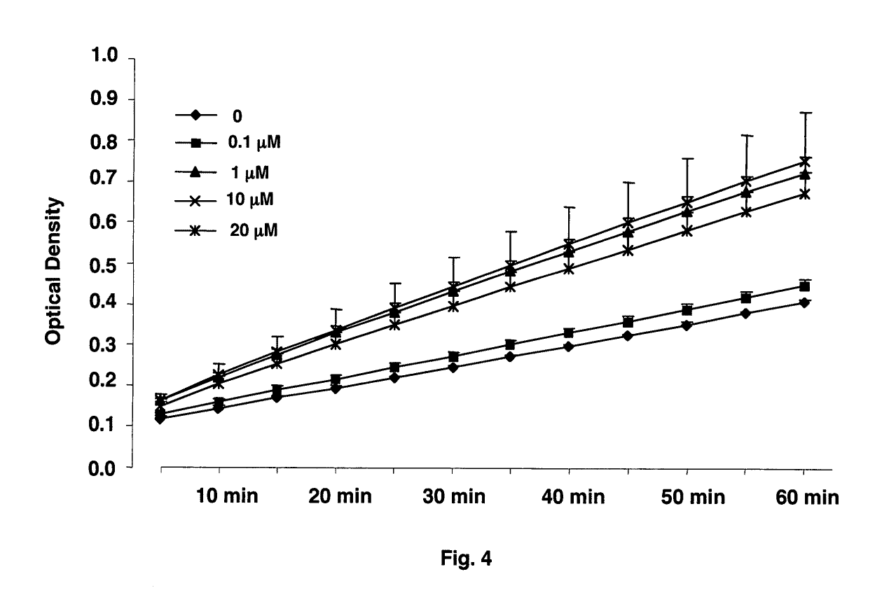

FIG. 4 shows that treatment of EA.hy926 cells with different concentrations of simvastatin for 24 hrs increases endothelial cell thrombomodulin activity in a dose-dependent manner. The dose-dependence is highly statistically significant (p<0.0001).

### Example 11

### The Effects of Statins on Normal and Irradiated Endothelial Cells

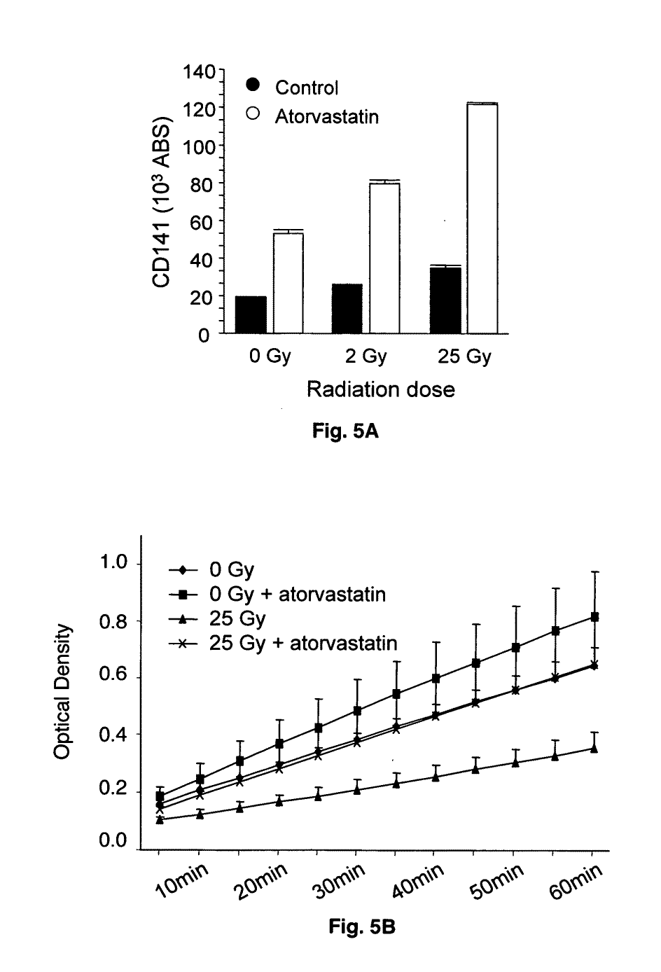

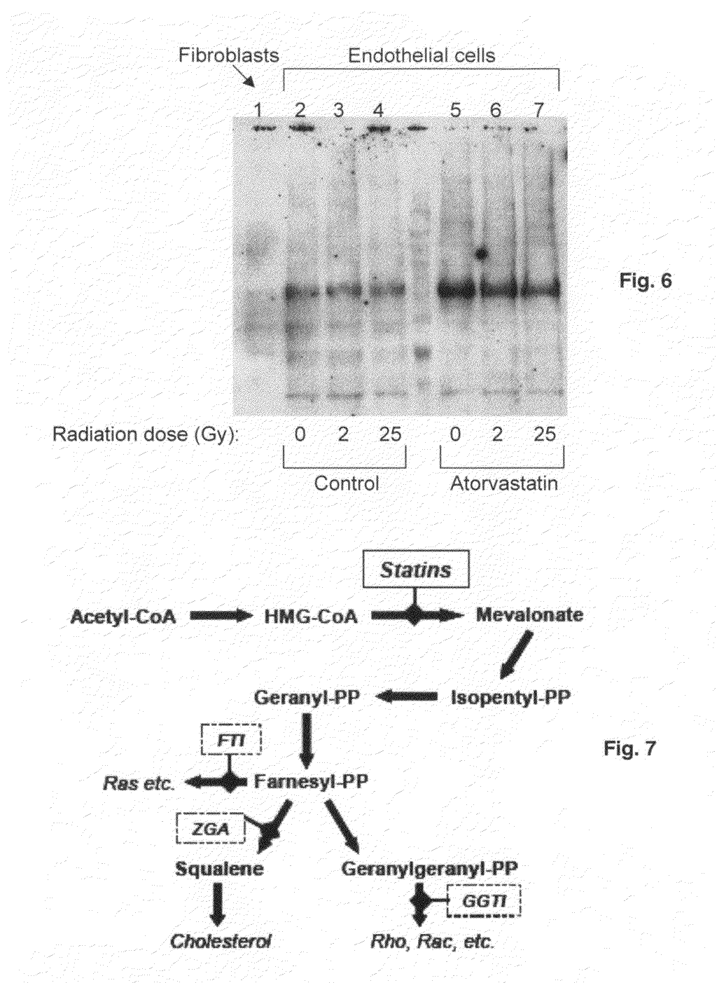

Treatment of EA.hy926 cells with atorvastatin before radiation showed that statin-treated endothelial cells had significantly higher thrombomodulin antigen expression and thrombomodulin activity compared to control endothelial cells, and that statin treatment essentially restored normal thrombomodulin activity in endothelial cells exposed to 25 Gy single dose radiation (FIGS. 5A-5B, 6).

Total thrombomodulin protein in cell lysates was measured by western blotting. EA.hy926 cells were grown to early (90%+) confluence. The cells were washed with 1×PBS twice and lysed by adding 1 ml lysis buffer (10 mM Tris, pH 7.4; 150 mM NaCl; 1 mM EDTA; 1% NP-40 or IGPAL CA630; 1 mM PMSF), and the lysate was collected into eppendorf tubes. Protein concentrations were measured with the BCA protein assay kit (Pierce). Protein aliquots of each sample were mixed with 4× loading buffer, placed in a boiling water bath for 5 min, and subsequently separated by NuPAGE 4-10% Bis-tris gel electrophoresis (Invitrogen Life Technologies). The proteins were transferred to a nitrocellulose membrane, probed with primary monoclonal mouse anti-human thrombomodulin antibody at 1:1000 and secondary anti-mouse IgG-HRP antibody at 1:2000 (American Diagnostica, Inc.) using standard procedures. The HRP signal was detected using ECL detection reagents (Amersham Life Sciences) and visualized on X-ray film.

### Example 12

### Atorvastatin Upregulates TM Expression and Activity via the Mevalonate Pathway by Depleting Geranylgeranyl-Pyrophosphate

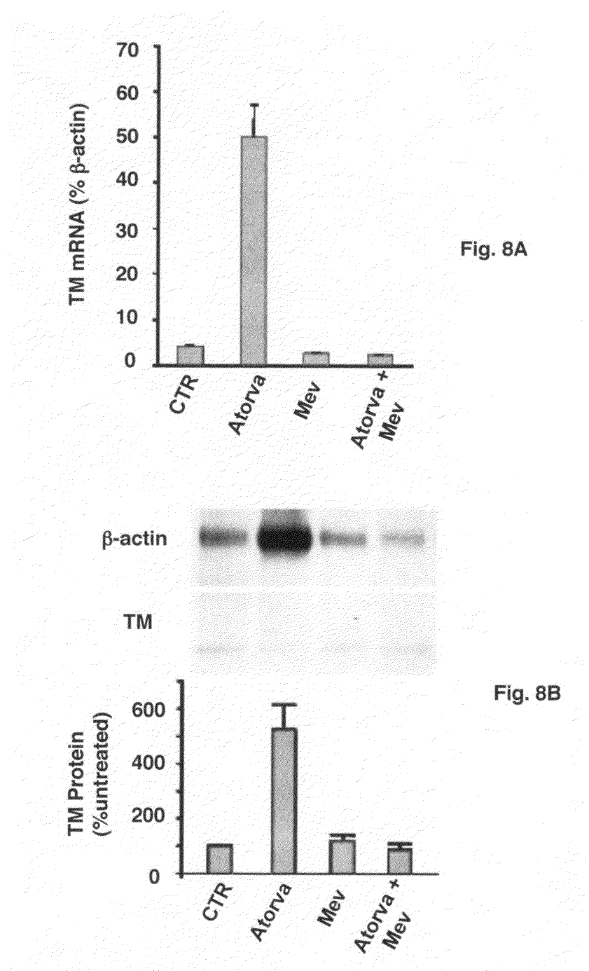

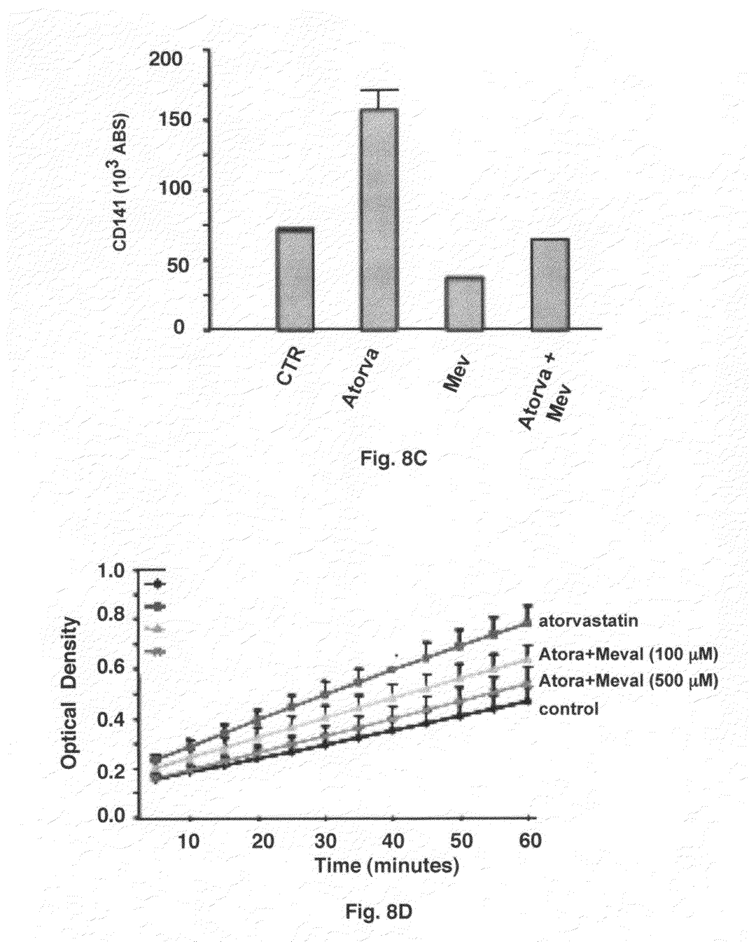

Statins, by inhibiting HMG-CoA reductase, reduce the formation of mevalonate (FIG. 7). Pre-incubation of cells with 100 mM or 500 mM mevalonate for 30 min prior to 24-hour exposure of EA.hy926 cells to 10 mM atorvastatin resulted in dose-dependent inhibition of atorvastatin-induced thrombomodulin upregulation, with virtually complete inhibition at the higher mevalonate concentration (FIGS. 8A-8D). These results demonstrate that the effect of atorvastatin on thrombomodulin expression is mediated via the mevalonate pathway. The effects of pre-incubating EAhy.926 cells for 30 min with mevalonate pathway intermediates (10 mM FPP, 10 mM GGPP, or 100 mM squalene) prior to atorvastatin exposure were also examined. GGPP completely blocked atorvastatin-induced thrombomodulin upregulation, whereas FPP only partially blocked upregulation, and squalene was ineffective in blocking these effects (Table 3).

Whether specific enzyme inhibitors (farnesyl transferase inhibitor I and II [FTI-I, FTI-II], geranylgeranyl transferase inhibitor-298 [GGTI-298, a GGTI-I inhibitor], and zaragozic acid [an inhibitor of squalene synthase, the final regulated enzyme in the cholesterol synthetic pathway]) influenced thrombomodulin activity in EA.hy926 cells was also examined. GGTI-298 mimicked atorvastatin by increasing thrombomodulin activity, whereas FTI and zaragozic acid had no effect (data not shown). These data show that atorvastatin upregulates endothelial cell thrombomodulin by a mechanism that involves GGPP depletion.

TABLE 3
Influence of mevalonate pathway intermediates on thrombomodulin expression
and activity in EA.hy926 endothelial cells (mean ± SEM).

| | | | | | | | A + | A + |
|---|---|---|---|---|---|---|---|---|
| | CTR | FPP | Squalene | GGPP | A | A + FPP | Squalene | GGPP |
| mRNA | 4.4 ± 0.2 | 8.2 ± 1.9 | 5.9 ± 1.0 | 5.4 ± 0.5 | 71.7 ± 8.8 | 29.3 ± 2.1 | 70.2 ± 10.2 | 8.9 ± 2.2 |
| Protein | 100 | 94 ± 9 | 147 ± 29 | 124 ± 11 | 403 ± 70 | 360 ± 55 | 333 ± 44 | 168 ± 35 |
| Antigen | 58.9 ± 1.5 | 74.3 ± 1.1 | 60.9 ± 1.9 | 66.6 ± 5 | 146.2 ± 1.9 | 126.4 ± 0.5 | 153.1 ± 10.7 | 77.0 ± 3.5 |
| Activity | 2.4 ± 0.3 | 3.6 ± 0.3 | 2.4 ± 0.6 | 3.0 ± 0.7 | 4.1 ± 0.3 | 5.3 ± 0.7 | 6.8 ± 0.8 | 3.0 ± 0.5 |

CTR = untreated control cells; FPP = farnesyl pyrophosphate 10 μM; Squalene = squalene 100 μM; GGPP = geranylgeranylpyrophosphate 10 μM; A = atorvastatin 10 μM.

### Example 14

### Atorvastatin Upregulates TM Activity by a Nitric Oxide-Dependent Mechanism

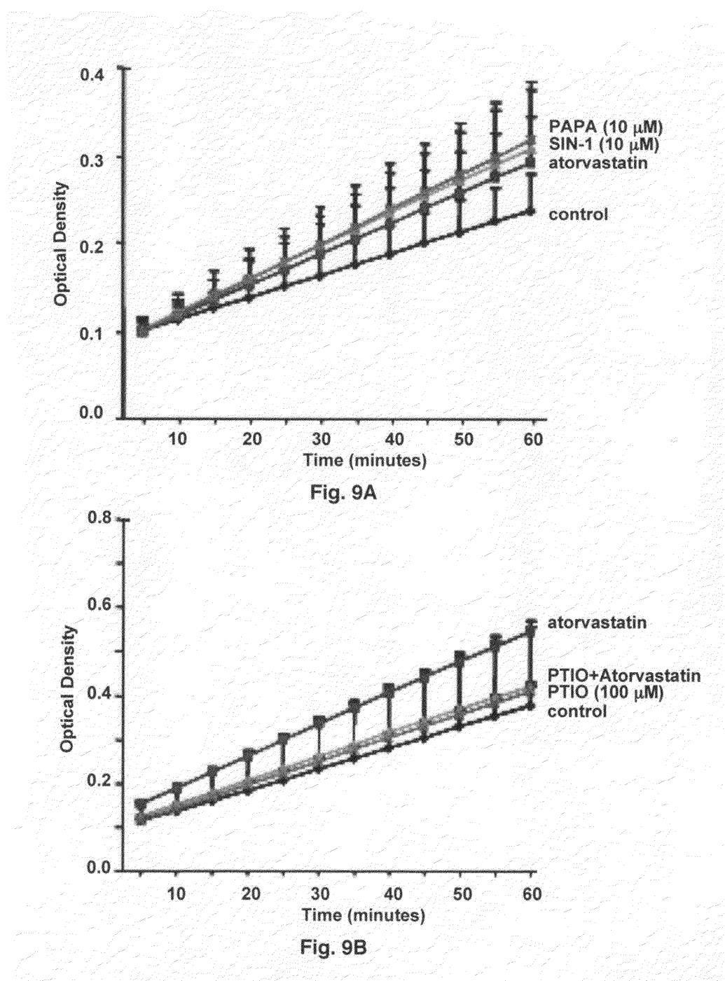

Statins increase the activity of endothelial nitric oxide synthase (NOS3) by a variety of mechanisms, and many of the vasculoprotective effects of statins are presumed to be mediated by nitric oxide (NO). Incubation of EA.hy926 cells with 10 mM SIN-1, a rapid NO donor, or 10 mM PAPA-NONOate, a slow NO donor, mimicked the effect of atorvastatin on thrombomodulin activity (FIG. 9A). Conversely, pre-incubation of cells with 100 mM PTIO, an NO scavenger, inhibited the atorvastatin-induced increase in thrombomodulin activity (FIG. 9B). These results suggest that increased NO production in response to atorvastatin may be responsible for the observed increase in thrombomodulin activity.

### Example 15

### Atorvastatin Counteracts TNF-a Induced Downregulation of Endothelial Cell TM Expression and Activity

TNF-a potently downregulates endothelial thrombomodulin, an effect that is pathophysiologically significant in sepsis and related disorders. Therefore, the effect of atorvastatin on TNF-a-induced downregulation of endothelial cell thrombomodulin was examined. Exposure of EA.hy926 cells to TNF-a (1-20 ng/ml for 24 hours) caused a dose-dependent increase in apoptosis (4-fold at 10 ng/ml) and tissue factor (2.5-fold) and decreased thrombomodulin by 75% (data not shown). EA.hy926 cells were pre-treated with atorvastatin (10 μM for 30 min) before exposure to TNF-a (1 or 10 ng/ml, 24 hours). Atorvastatin completely counteracted the effect of TNF-a on endothelial cell thrombomodulin activity and raised the levels of thrombomodulin gene expression, protein, and surface thrombomodulin expression significantly above those of untreated control cells as in Table 4. In contrast, atorvastatin did not affect TNF-a-induced tissue factor expression or apoptosis (data not shown).

TABLE 4
Atorvastatin counteracts the negative effect of TNF-a on
TM expression and activity in EA.hy926 endothelial cells

| | CTR | TNF (1) | A + TNF (1) | TNF (10) | A + TNF (10) |
|---|---|---|---|---|---|
| mRNA | 5.9 ± 1.6 | 2.4 ± 1.0 | 26.1 ± 3.7 | 1.9 ± 0.5 | 9.9 ± 0.4 |
| Protein | 100 | 91 ± 27 | 402 ± 23 | 38 ± 16 | 463 ± 63 |
| Antigen | 70.0 ± 1.0 | 37.3 ± 1.5 | 117.7 ± 2.6 | 25.9 ± 0.6 | 79.2 ± 2.7 |
| Activity | 8.2 ± 0.4 | 5.3 ± 0.2 | 8.8 ± 0.4 | 4.7 ± 0.2 | 7.3 ± 0.2 |

(means and standard errors). CTR = untreated control cells; TNF(1) = TNF-a 1 ng/ml; TNF(10) TNF- a 10 ng/ml; A = atorvastatin 10 μM.

The finding that statin counteracts the negative effect of TNF-a on endothelial cell thrombomodulin suggests the potential use of statin as an adjuvant in patients with sepsis and related disorders. During sepsis, the vascular endothelium is strongly pro-coagulant, due to decreased expression of thrombomodulin and possibly to increased expression of tissue factor. Despite longstanding interest in therapeutic modulation of the coagulation system in sepsis, the only approach to date that has translated into a survival benefit in phase III clinical trials is the administration of recombinant activated protein C (Bernard et al., 2001). Importantly, this trial showed that activated protein C infusion was equally beneficial in patients with normal and low protein C levels, suggesting that the critical factor is not reduced availability of protein C, but a defective activation mechanism. Statins could possibly be used to increase endothelial thrombomodulin and protein C activation in patients at risk for or with established sepsis, thereby providing an inexpensive and safe prophylactic or therapeutic intervention for restoring the anticoagulant properties of the endothelium. In support of this notion, an intriguing clinical study showed that patients who were on statin therapy when they developed sepsis were 7 times less likely to die than patients who were not on statin therapy (Liappis et al., 2001).

This study demonstrates that statin strongly upregulates endothelial cell thrombomodulin expression and activity. These findings represent a new and potentially important pleiotropic effect of statins and point to future use of statins as a possible prophylactic or therapeutic intervention in disorders associated with deficient endothelial thrombomodulin or deficient protein C activation.

### Example 16

### Effect of ATORVASTATIN™ and SIMVASTATIN™ on Endothelial Thrombomodulin Activity (Protein C Activation Assay) in Human Intestinal Microvascular Cells

Radiation induces a deficiency in endothelial thrombomodulin in intestinal microvasculature and this may be a key to the development of intestinal radiation toxicity. The present invention demonstrates that statins would be beneficial in treating intestinal disorders associated with reduced thrombomodulin. Statins, therefore, would be useful in treating any type of radiation injury. More broadly, the present invention demonstrates the effect of statins on endothelial thrombomodulin and that this effect is seen in most or all vascular beds.

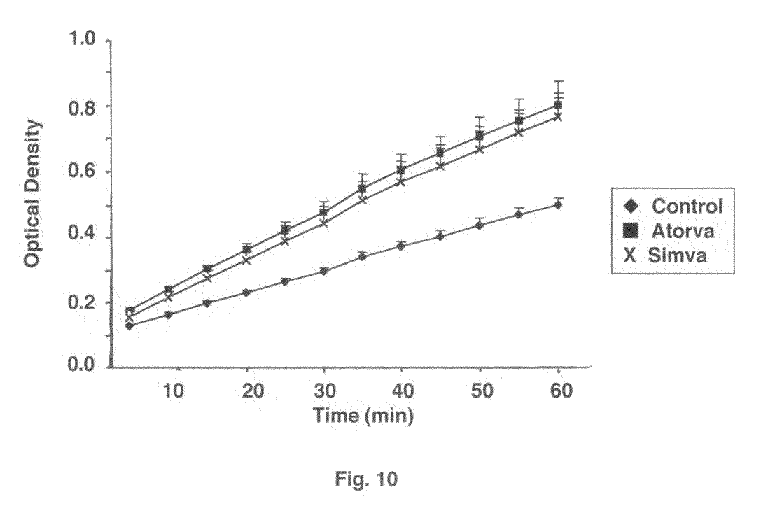

FIG. 10 shows that both ATORVASTATIN™ and SIMVASTATIN™ significantly affected endothelial thrombomodulin activity (protein C activation assay) in human intestinal microvascular cells.

### Example 17

### In Vivo Data

The following in vivo data supports the in vitro data described above. A 10 cm loop of mouse small intestine was exposed to 18.5 or 20.0 Gy single dose irradiation. Mice were treated with simvastin (50 mg/kg/day p.o.) beginning 2 weeks before irradiation 15 and continuing for 2 weeks after irradiation. Compared to control mice, simvastin reduced radiation induced mortality from 70% to 9% (p<0.01) in the 20 Gy group and conferred highly statistically significant protection against structural radiation injury (p<0.01). Compared to control mice, simvastin reduced radiation-induced mortality from 70% to 9% (p<0.01).

These data provide strong support for the beneficial effect of stains in in vivo radiation injury, an effect which may be a result of upregulation of endothelial thrombomodulin. Thus, statins would be of significant benefit if given during and after radiation therapy, e.g., of cancer. Thus, statins could be used to ameliorate the side effects from normal tissue injury.

In addition, the present invention provides in another embodiment that statins would be of significant benefit as a treatment for an individual exposed to non-therapeutic ioninizing radiation. Hence, statins would also be useful in treating individuals involved in radiation accidents, nuclear warfare, radiation terrorism such as “dirty bombs” as well as other situations associated with radiation exposure, e.g., space flights.

The following references are cited herein:
Albertson et al. Blood Coagul Fibrinolysis, 12:729-733 (2001).
Bernard et al. N Engl J. Med. 344:699-709 (2001).
Faust et al. N Engl J Med. 345:408-416 (2001).
Fink et al. Int J Dev Biol, 37:221-226 (1993).
Healy et al. Proc. Natl. Acad. Sci. USA 92:850-854 (1995).
Richter et al. Radiother Oncol, 44:65-71 (1997).
Richter et al. Am J Surg, 176:642-647 (1998).
Wang et al. Am J Pathol, 160:2063-2072 (2002).
Warren et al. JAMA 286:1869-1878 (2001).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method for decreasing mortality of an individual from an ionizing radiation injury, comprising:
administering a 3-hydroxy-3-methylglutaryl coenzyme A reductase inhibitor to the individual one or more times to ameliorate the effects of radiation exposure, thereby reducing mortality of the individual.

2. The method of claim 1, wherein the 3-hydroxy-3-methylglutaryl coenzyme A reductase inhibitor is pravastatin or its sodium salt, simvastatin, lovastatin, atorvastatin, rosuvastatin, or fluvastatin.

3. The method of claim 1, wherein the injury is radiation enteropathy.

4. The method of claim 1, wherein the 3-hydroxy-3-methylglutaryl coenzyme A reductase inhibitor is administered one or more of a time period that is before, during or after radiation exposure.

5. The method of claim 1, wherein said inhibitor is administered orally or parenterally.

6. A method for decreasing mortality in an individual with a disorder associated with endothelial dysfunction, comprising:
administering a 3-hydroxy-3-methylglutaryl coenzyme A reductase inhibitor to the individual one or more times as to increase thrombomodulin expression and function in the endothelia of the individual, thereby decreasing mortality.

7. The method of claim 6, wherein the 3-hydroxy-3-methylglutaryl coenzyme A reductase inhibitor is pravastatin or its sodium salt, simvastatin, lovastatin, atorvastatin, rosuvastatin, or fluvastatin.

8. The method of claim 6, wherein the disorder is radiation enteropathy, sepsis or adult respiratory distress syndrome.

9. The method of claim 6, wherein the individual is at risk of developing the disorder associated with endothelia dysfunction and the 3-hydroxy-3-methylglutaryl coenzyme A reductase inhibitor is administered when the risk is determined.

10. The method of claim 9, wherein the risk is developing radiation enteropathy from therapeutic ionizing radiation, said 3-hydroxy-3-methylglutaryl coenzyme A reductase inhibitor administered at least prior to radiation exposure.

11. The method of claim 6, wherein said inhibitor is administered orally or parenterally.

12. A method of increasing cell surface thrombomodulin expression and function in an endothelial cell, comprising:
contacting the cell with a 3-hydroxy-3-methylglutaryl coenzyme A reductase inhibitor, thereby increasing thrombomodulin expression and function.

13. The method of claim 12, wherein the 3-hydroxy-3-methylglutaryl coenzyme A reductase inhibitor is pravastatin or its sodium salt, simvastatin, lovastatin, atorvastatin, rosuvastatin, or fluvastatin.

14. The method of claim 12, wherein the endothelial cell is contacted in vitro or in vivo.

15. The method of claim 12, wherein the endothelial cell is contacted in vivo prior to, during or after exposure to ionizing radiation.

16. The method of claim 12, wherein the endothelial cell is contacted in vivo in an individual having a disorder associated with endothelial dysfunction.

17. The method of claim 16, wherein the disorder is radiation enteropathy, sepsis or adult respiratory distress syndrome.

18. The method of claim 16, wherein the 3-hydroxy-3-methylglutaryl coenzyme A reductase inhibitor decreases mortality of the individual.

19. The method of claim 16, wherein contact is via oral or parenteral administration.
